(19) United States
(12) Patent Application Publication
(10) Pub. No.: US 2018/0108441 A1
(43) Pub. Date: Apr. 19, 2018

(54) System For Searching Data Structures Stored In A Database

(71) Applicant: Ali Adel Hussam, Columbia, MO (US)

(72) Inventor: Ali Adel Hussam, Columbia, MO (US)

(21) Appl. No.: 15/673,168

(22) Filed: Aug. 9, 2017

Related U.S. Application Data
(63) Continuation of application No. 14/029,584, filed on Sep. 17, 2013, now abandoned.

(51) Int. Cl. G06Q 50/24

(52) U.S. Cl. CPC G16H 50/70

(57) ABSTRACT

A computer-implemented method includes transmitting information indicative of an outcome questionnaire; receiving, from the first client device, answers to the series of questions; generating, based on the answers, an outcome score that is indicative of the severity of the functional limitation; determining a modifier that is indicative of a range of impairment, limitation or restriction of the portion of the user's anatomy; receiving a request to generate an insurance report, with the request comprising a selected type of functional limitation and a visit type; identifying a code that is indicative of the functional limitation that is being treated by the service provider and that is indicative of a point in time in a course of treatment of the user; generating, based on the identified code and the modifier, the insurance report; and submitting the insurance report to an insurance provider.

### CLAIM OF PRIORITY

This application is a continuation of and claims priority under 35 U.S.C. § 120 to U.S. patent application Ser. No. 14/029,584, filed Sep. 17, 2013, the entire contents of each of which are hereby incorporated by reference.

### BACKGROUND

Processing insurance claims can be a difficult and convoluted process. Certain insurance providers request that certain information be provided at various intervals based on a medical patient's recovery protocol.

### TECHNICAL FIELD

The present disclosure is directed towards a system for searching data structures stored in a database and for processing a plurality of structured data items with a rules engine that applies programmed logic to one or more data structures identified from the searching and to one or more of the structured data items in the plurality.

### SUMMARY

In general, generating an insurance claim reports is described. In general, when a patient visits a service provider (e.g., a physical therapist, occupational therapist, hospital, clinic, or other medical service provider), the medical patient may be asked one or more questions regarding a functional limitation or restriction with respect to a part of the medical patient's anatomy. In an example, if the medical patient undergoes a knee operation, the medical patient may be asked a number of questions directed to range of motion limitations in the knee, strength limitations in the knee, stability limitations in the knee and so forth. The answers to these questions can be used to determine an outcome score for the medical patient.

In an aspect of the present disclosure, a computer-implemented method includes transmitting, to a first client device of a user, information indicative of an outcome questionnaire, with the outcome questionnaire comprising a series of questions to assess a severity of a functional limitation of a portion of the user's anatomy; receiving, from the first client device, answers to the series of questions; generating, based on the answers, an outcome score that is indicative of the severity of the functional limitation; determining, at least partly based on the outcome score, a modifier that is indicative of a range of impairment, limitation or restriction of the portion of the user's anatomy; receiving, from a second client device of a service provider, a request to generate an insurance report, with the request comprising a selected type of functional limitation and a visit type; identifying, based on contents of the received request, a code that is indicative of the functional limitation that is being treated by the service provider and that is indicative of a point in time in a course of treatment of the user; generating, based on the identified code and the modifier, the insurance report; and submitting the insurance report to an insurance provider. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Implementations of the disclosure may include one or more of the following actions. In some implementations, the actions include accessing a rules engine that comprises a plurality of rules for generating modifiers, with the rules engine including a plurality of rules specifying relationships among outcome scores and modifiers; causing an execution of the rules engine; applying, based on execution of the rules engine, one or more of the rules in the plurality to the generated outcome score; and identifying, based on applying, the modifier.

The actions also include accessing information indicative of a mapping of items of code selection information to a plurality of codes; wherein an item of code selection information comprises: information specifying a type of functional limitation; and information specifying a visit type; determining a match between (i) contents of the item of code selection information, and (ii) the selected type of functional limitation and the visit type specified in the request; wherein identifying the code comprises: identifying, based on the determined match, a code associated with the matching item of code selection information.

The actions include generating, based on the answers, the outcome score that is indicative of the severity of the functional limitation comprises: accessing a rules engine that comprises a plurality of rules for generating outcome scores, based on answers to questions included in outcome questionnaires; causing an execution of the rules engine; applying, based on execution of the rules engine, one or more of the rules in the plurality to the received answers; and generating, based on applying, the outcome score. The functional limitation comprises at least one of: a mobility functional limitation; a changing and maintaining body position functional limitation; a carrying, moving and handling objects functional limitation; a self care functional limitation; a other physical or occupational primary functional limitation; a other physical or occupational subsequent functional limitation; a swallowing functional limitation; a motor speech functional limitation; a spoken language comprehension functional limitation; a spoken language expression functional limitation; an attention functional limitation; a memory functional limitation; a voice functional limitation; and a other speech language pathology functional limitation. The code is a G-code. The G-code is at least one of: a mobility G-code of G8978 for mobility: walking & moving around functional limitation, current status, at therapy episode outset and at reporting intervals; a mobility G-code of G8979, for mobility: walking & moving around functional limitation, projected goal status, at therapy episode outset, at reporting intervals, and at discharge or to end reporting; a mobility G-code of G8980, for mobility: walking & moving around functional limitation, discharge status, at discharge from therapy or to end reporting; a changing and maintaining body position G-code of G8981, for changing and maintaining body position functional limitation, current status, at therapy episode outset and at reporting intervals; a changing and maintaining body position G-code of G8982, for changing and maintaining body position functional limitation, projected goal status, at therapy episode outset, at reporting intervals, and at discharge or to end reporting; a changing and maintaining body position G-code of G8983, for changing and maintaining body position functional limitation, discharge status, at discharge from therapy or to end reporting; a carry, moving, and handling objects G-code of G8984, for carrying, moving and handling objects functional limitation, current status, at therapy episode outset and at reporting intervals; a carry, moving, and handling objects G-code of G8985, for carrying, moving and handling objects functional limitation, projected goal status, at therapy episode outset, at reporting intervals, and at discharge or to end reporting; a carry, moving, and handling objects G-code of G8986 for carrying, moving and handling objects a self care G-code of G8987, for self care functional limitation, current status, at therapy episode outset and at reporting intervals; a self care G-code of G8988, for self care functional limitation, projected goal status, at therapy episode outset, at reporting intervals, and at discharge or to end reporting a self care G-code of G8989, for self care functional limitation, discharge status, at discharge from therapy or to end reporting; a other physical or occupational primary G-code of G8990, for other physical or occupational primary functional limitation, current status, at therapy episode outset and at reporting intervals; a other physical or occupational primary G-code of G8991, for other physical or occupational primary functional limitation, projected goal status, at therapy episode outset, at reporting intervals, and at discharge or to end reporting; a other physical or occupational primary G-code of G8992, for other physical or occupational primary functional limitation, discharge status, at discharge from therapy or to end reporting; a other physical or occupational subsequent G-code of G8993, for other physical or occupational subsequent functional limitation, current status, at therapy episode outset and at reporting intervals; a other physical or occupational subsequent G-code of G8994, for other physical or occupational subsequent functional limitation, projected goal status, at therapy episode outset, at reporting intervals, and at discharge or to end reporting; a other physical or occupational subsequent G-code of G8995, for other physical or occupational subsequent functional limitation, discharge status, at discharge from therapy or to end reporting; a swallowing G-code of G8996, for swallowing functional limitation, current status at time of initial therapy treatment/episode outset and reporting intervals; a swallowing G-code of G8997, for swallowing functional limitation, projected goal status, at initial therapy treatment/outset and at discharge from therapy; a swallowing G-code of G8998, for swallowing functional limitation, discharge status, at discharge from therapy/end of reporting on limitation; a motor speech G-code of G8999, for motor speech functional limitation, current status at time of initial therapy treatment/episode outset and reporting intervals; a motor speech G-code of G9186, for motor speech functional limitation, projected goal status at initial therapy treatment/outset and at discharge from therapy; a motor speech G-code of G9158, for motor speech functional limitation, discharge status at discharge from therapy/end of reporting on limitation; a spoken language comprehension G-code of G9159, for spoken language comprehension functional limitation, current status at time of initial therapy treatment/episode outset and reporting intervals; a spoken language comprehension G-code of G9160, for spoken language comprehension functional limitation, projected goal status at initial therapy treatment/outset and at discharge from therapy; a spoken language comprehension G-code of G9161, for spoken language comprehension functional limitation, discharge status at discharge from therapy/end of reporting on limitation; a spoken language expressive G-code of G9162, for spoken language expression functional limitation, current status at time of initial therapy treatment/episode outset and reporting intervals; a spoken language expressive G-code of G9163, for spoken language expression functional limitation, projected goal status at initial therapy treatment/outset and at discharge from therapy; a spoken language expressive G-code of G9164, for spoken language expression functional limitation, discharge status at discharge from therapy/end of reporting on limitation; an attention G-code of G9165, for attention functional limitation, current status at time of initial therapy treatment/episode outset and reporting intervals; an attention G-code of G9166, for attention functional limitation, projected goal status at initial therapy treatment/outset and at discharge from therapy; an attention G-code of G9167, for attention functional limitation, discharge status at discharge from therapy/end of reporting on limitation; a memory G-code of G9168, for memory functional limitation, current status at time of initial therapy treatment/episode outset and reporting intervals; a memory G-code of G9169, for memory functional limitation, projected goal status at initial therapy treatment/outset and at discharge from therapy; a memory G-code of G9170, for memory functional limitation, discharge status at discharge from therapy/end of reporting on limitation; a voice G-code of G9171, for voice functional limitation, current status at time of initial therapy treatment/episode outset and reporting intervals; a voice G-code of G9172, for voice functional limitation, projected goal status at initial therapy treatment/outset and at discharge from therapy; a voice G-code of G9173, for voice functional limitation, discharge status at discharge from therapy/end of reporting on limitation; a other speech language pathology G-code of G9174, for other speech language pathology functional limitation, current status at time of initial therapy treatment/episode outset and reporting intervals; a other speech language pathology G-code of G9175, for other speech language pathology functional limitation, projected goal status at initial therapy treatment/outset and at discharge from therapy; and a other speech language pathology G-code of G9176, for other speech language pathology functional limitation, discharge status at discharge from therapy/end of reporting on limitation.

In some implementations, the modifier comprises a value of one or more of: CH; CI; CJ; CK; CL; CM; and CN. In other implementations, the range of impairment, limitation or restriction is one or more of: 0%; at least 1% but less than 20%; at least 20% but less than 40%; at least 40% but less than 60%; at least 60% but less than 80%; at least 80% but less than 100%; and 100%. The outcome score comprises a first outcome score, and wherein the generated insurance report further comprises: a first visual representation of a timeline that specifies at least two visits of the user to the service provider; a second visual representation of the first outcome score for the outcome questionnaire at a first one of the at least two visits; and a third visual representation of a second outcome score for the outcome questionnaire at a second one of the at least two visits. The insurance report comprises a claim submission. The visit type comprises one or more of an initial visit, a tenth visit and a discharge visit. The actions include receiving, from the first client device, information indicative of the portion of the user's anatomy in which the user is experiencing the functional limitation; and selecting, from a plurality of outcome questionnaires and based on the received information, the outcome questionnaire that is submitted to the first client device, with the selected outcome questionnaire including questions that pertain to the portion of the user's anatomy in which the user is experiencing the functional limitation.

All or part of the foregoing may be implemented as a computer program product including instructions that are stored on one or more non-transitory machine-readable storage media, and that are executable on one or more processing devices. All or part of the foregoing may be implemented as an apparatus, method, or electronic system that may include one or more processing devices and memory to store executable instructions to implement the stated functions.

The details of one or more implementations are set forth in the accompanying drawings and the description below. Other features, objects, and advantages will be apparent from the description and drawings, and from the claims.

### BRIEF DESCRIPTION OF THE FIGURES

FIG. 1 is a block diagram of components of a system used by a service provider.

FIG. 2 shows a hand held device executing an application that generates a one or more graphical user interfaces that can be used to interface with aspects of the system used by the service provider.

FIG. 3-5 are screen images of graphical users interfaces generated by one or more applications that can be used to interface with aspects of the system used by the service provider.

FIG. 6 illustrates an example report that can be generated by the system used by the service provider.

FIG. 7 is a flow chart showing an example process for generating and submitting one or more insurance reports.

Like reference symbols in the various drawings indicate like elements.

### DETAILED DESCRIPTION

In general, generating an insurance claim report (e.g., a G-code report) is described. In general, when a patient visits a service provider (e.g., a physical therapist, occupational therapist, hospital, clinic, or other medical service provider), the user (e.g., medical patient) may be asked one or more questions regarding a functional limitation or restriction with respect to a part of the medical patient's anatomy. In an example, if the user undergoes a knee operation, the user may be asked a number of questions directed to range of motion limitations in the knee, strength limitations in the knee, stability limitations in the knee and so forth. The answers to these questions can be used to determine an outcome score for the user.

In general, the medical industry has a number of standardized outcome questionnaires that can be used to generate outcome scores for multiple users using multiple service providers. In an example, the outcome scores may be predictable in that similar answers for a particular questionnaire will generally yield a same or similar outcome score.

Based on these predictable outcome scores, insurance providers and other service providers have generated certain codes (e.g., modifiers) that are indicative of the functional limitation and that are based on the particular outcome scores. In some implementations, insurance providers use these determined codes to track the progress of the user and/or as a mechanism for paying insurance claims. In an example, if a user has a first outcome score during a first consultation and at the end of a rehabilitation regimen has a worse outcome score relative to the first consultation, an insurance provider may be less likely to pay the medical fees because the prescribed treatment did not have the desired effect of improving the user's condition. In some implementations, insurance providers may request or otherwise require a report that includes one or more codes indicative of the one or more functional limitations that are being treated. In some implementations, the insurance providers may request or otherwise require that these reports be submitted at various predetermined intervals. In a particular example, the insurance providers may require that a report be submitted by a service provider after a first consultation, after a tenth consultation, and when a user is discharged from the care of the service provider.

In general, the modifiers determined from the outcome scores can be ascertained at an initial visit. Based on these ascertained modifiers and the knowledge and experience of the service provider, a goal modifier that corresponds to a goal range of impairment can be defined for a particular limitation. As the user attends therapy sessions or performs other actions associated with a recovery regimen, the service provider may periodically re-assess the user's outcome score (and by extension determine a modifier indicative of a range of impairment). In some implementations, the service provide does not discharge the user until the user's matches the goal modifier, even if the user has finished the initial number of prescribed therapy sessions.

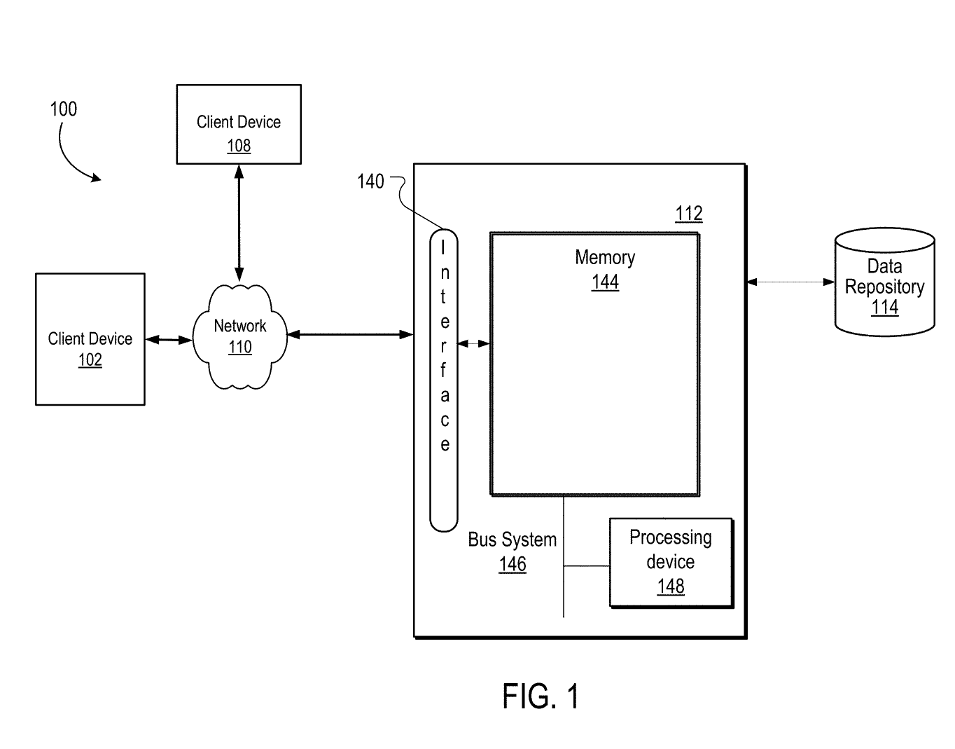

In general, FIGS. 1-7 are described in relation to the collection and use of certain personal medical information of one or more users. The processes and procedures used by the service provider described herein for the collection and use of the personal medical information are compliant with the Health Insurance Portability and Accountability Act (HIPPA) and any other relevant regulations for the protection of personal medical information and other information. In addition, in some implementations, aspects of some or all of the information that is collected by the service provider can be anonymized to add additional privacy protections for the users of the system.

FIG. 1 is a block diagram of components of a system 100 that can be operated by a service provider. Again, the service provider can be some combination of a physical therapist, an occupational therapist, a hospital, a clinic, and one or more physicians, nurses, and other medical service professionals that provide medical care to one or more users. In FIG. 1, client devices 102, 108 can be any sort of computing devices capable of taking input from a user and communicating over network 110 with server 112 and/or with other client devices. For example, client devices 102, 108 can be mobile devices, desktop computers, laptops, cell phones, personal digital assistants (“PDAs”), servers, embedded computing systems, and so forth. In a particular example, the client devices 102 and 108 are tablet computing devices or other computing devices that include a touch-sensitive input-and-display device that allows a user using the client devices 102 and 108 to provide answers by touching appropriate regions of a presented outcome questionnaire. In an example, one or more of client devices 102, 108 can be used by an insurance company for receipt of an insurance report through a network.

In an example, the client devices 102 and 108 are configured to present one or more graphical users interfaces to their respective users. In a particular example, the client devices 102 and 108 have been installed with an application or other executable code. The application or executable code can cause presentation of the graphical user interfaces when the application or executable code is executed, e.g., by a processing device 148. These graphical user interfaces allow users to enter information and interact with other users of the health care entity system 100 and to provide ratings of one or more medical providers that are associated with the health care entity. Particular examples of graphical user interfaces are described in more detail below.

Server 112 can be any of a variety of computing devices capable of receiving data, such as a server, a distributed computing system, a desktop computer, a laptop, a cell phone, a rack-mounted server, and so forth. Server 112 may be a single server or a group of servers that are at a same location or at different locations.

The illustrated server 112 can receive data from client devices 102, 108 via input/output (“I/O”) interface 140. I/O interface 140 can be any type of interface capable of receiving data over a network, such as an Ethernet interface, a wireless networking interface, a fiber-optic networking interface, a modem, and so forth. Server 112 also includes a processing device 148 and memory 144. A bus system 146, including, for example, a data bus and a motherboard, can be used to establish and to control data communication between the components of server 112.

The illustrated processing device 148 may include one or more microprocessors. Generally, processing device 148 may include any appropriate processor and/or logic that is capable of receiving and storing data, and of communicating over a network (not shown). Memory 144 can include a hard drive and a random access memory storage device, such as a dynamic random access memory, or other types of non-transitory machine-readable storage devices. Memory 144 stores computer programs (not shown) that are executable by processing device 148 to perform the techniques described herein. In a particular example, the memory can store an insurance report generator that can generate reports based on information indicative of a severity of a functional limitation of a user's anatomy. In an example, the generated report can be used submitted to one or more insurance providers to collect payment for medical servers rendered to the user.

In an example, server 112 generates and implements an insurance report generator. In an example, the server 112 can receive outcome questionnaires composed by service providers or other medical professionals. In a particular, one or more stock outcome questionnaires (e.g., a QuickDASH questionnaire, a LEFS questionnaire, an NDI questionnaire, an ODI questionnaire, and so forth) can be stored by the server 112. At some future time (e.g., when a user visits the service provider), the server 112 can provide the outcome questionnaires to the client devices 102 and 108. As the user completes the outcome questionnaire, the server 112 can receive medical answers from the client devices 102 and 108. In response, the server 112 can generate an outcome score based at least in part on the answers and store the outcome score in the data repository 114. Additional aspects of the server 112 are described elsewhere in this specification.

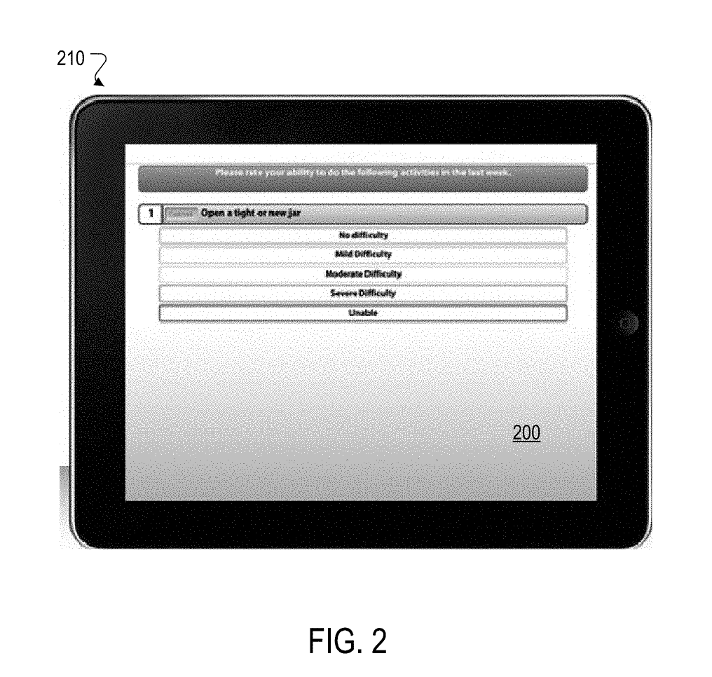

Referring now to FIG. 2, a screen image of a graphical user interface 200 is being shown as part of an application loaded on a client device 102 or 108, such as a tablet device 210. In the depicted example, the graphical user interface 200 can provide one or more outcome questions in the form of an outcome questionnaire. In general, the outcome questionnaire is tailored to a particular medical condition (or collection of medical conditions) for the user of the tablet device 210 (e.g., a user visiting the service provider) or reason for visiting the service provider.

In a particular example, if the user's visit to the service provider pertains to an issue with an upper extremity of the user's anatomy, a QuickDASH outcome questionnaire may be provided to the user. As another particular example, if the user's visit to the service provider pertains to an issue with a lower extremity of the user's anatomy, a LEFS outcome questionnaire may be provided to the user. As another particular example, if the user's visit to the service provider pertains to an issue with a cervical region of the user's anatomy, an NDI outcome questionnaire may be provided to the user. As another particular example, if the user's visit to the service provider pertains to an issue with a lumbar region of the user's anatomy, an ODI outcome questionnaire may be provided to the user. Other outcome questionnaires may also be provided.

In some implementations, the graphical user interface 200 may be customized to fit a smaller screen of the handheld device 210 relative to other client device 102 or 108, such as a desktop computer, laptop computer, and so forth. In some implementations, the answers to the questions may be color-coded to reflect a severity of a functional limitation to a particular portion of the user's anatomy. In an example, a red color indicates a highest severity of a functional limitation while a green color indicates a lowest severity of a functional limitation.

In general, the user can select one or more answers that correspond to questions of the outcome questionnaire. These answers can be used to generate an outcome score. In some implementations, and depending on the particular outcome questionnaire that is provided, a higher outcome score is indicative of a higher severity of functional limitation. In other implementations, and depending on the particular out questionnaire that is provided, a lower outcome score is indicative of a higher severity of functional limitation. Examples of different outcome scores based on different outcome questionnaires are described in more detail below.

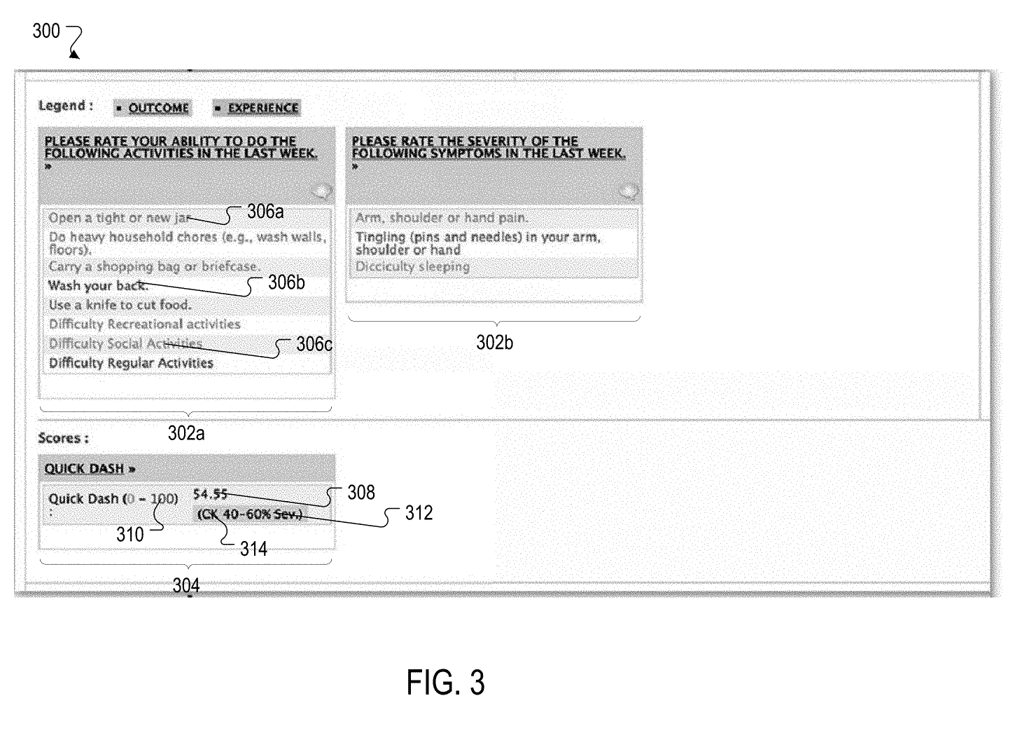

FIG. 3 is a screen image of a graphical user interface 300 that can present information indicative of answers to one or more outcome questionnaires. In general, the graphical user interface 300 can present one or more answers, an outcome score associated with a particular outcome questionnaire, and a determined severity modified to a medical profession (e.g., a physical or occupational therapist). In some implementations, the graphical user interface 300 can update in real-time as the user provides answers. In an example, as additional answers are received by a system (such as server 112), the outcome score and/or severity modifier can be updated to reflect the additional answers.

In general, the graphical user interface 300 can include one or more graphical regions that can be used to present information indicative of answers to one or more outcome questionnaires that are provided to a user (e.g., a medical patient using tablet device 210 (FIG. 2) to answer one or more question). In an example, the graphical user interface 300 can include one or more outcome regions 302a and 302b, and an outcome scores region 304.

In general, the outcome regions 302a and 302b present information indicative of one or more outcomes. In an example, the outcome regions 302a and 302b can present information indicative of one or more answers to questions. In some implementations, these questions can be organized or otherwise grouped in such a way to present information indicative of an outcome based on a particular theme or subject. In a particular example, the outcome region 302a presents information indicative of an outcome for activities that the user performed last week. In another particular example, the outcome region 302b presents information indicative of an outcome based on a severity of one or more possible symptoms.

In some implementations, the outcome regions 302a and 302b present the answers to the one or more questions and in a corresponding color that is indicative of a severity of functional limitation for a portion of the user's anatomy. In a particular example, the outcome region 302a includes an answer 306a “Open a tight or new jar” in a green colored-text that is indicative of a positive outcome. As another particular example, the outcome region 302a includes an answer 306b “Wash your back” in a red colored-text that is indicative of negative outcome. As yet another particular example, the outcome region 302a includes an answer 306c “Difficulty Social Activities” in a greenish-yellow colored text that is indicative of a slightly positive outcome (e.g., that is less positive than the green-colored text, but more positive than the red-colored text).

In general, the outcome scores region 304 can present information indicative of one or more outcomes scores for particular outcome questionnaires. In a particular example, the outcome scores region 304 includes an outcome score 308 for a “Quick Dash” (or a QuickDASH) outcome questionnaire. The outcome scores region 304, however, can include many other outcome scores instead of or in addition to the outcome score 308 based on how many outcome questionnaires the particular user has answered or is answering. In some implementations, each of the outcome questionnaires for which outcome scores are provided can be associated with an outcome scale that is presented with the name of the outcome questionnaire and the outcome score. In a particular example, the outcome scores region 304 can include an outcome scale 310 that indicates that a score of “0” for the “Quick Dash” outcome questionnaire is the most favorable outcome score (e.g., because the “0” is presented as a green-colored text) and that a score of “100” for the “Quick Dash” outcome questionnaire is the least favorable outcome score (e.g., because the “100” is presented as a red-colored text).

In some implementations, the outcome scores region 304 can also present information indicative of range of impairment, limitation, or restriction of the portion of the user's anatomy in which the outcome questionnaire is directed. In an example, the outcome scores region 304 can present a modifier indicative a range of impairment, limitation, or restriction of the portion of the user's anatomy. In a particular example, the outcome scores region presents both a range of impairment 312 (e.g., “40-60% Sev,” indicating a 40-60% limitation of the lower extremities based on the outcome score of the “Quick Dash” outcome questionnaire) and a modifier 314 (e.g., “CK”) for the range of impairment. In some implementations, the server 112 (FIG. 1) can access a rules engine that can convert an outcome score to a range of impairment and/or to a modifier.

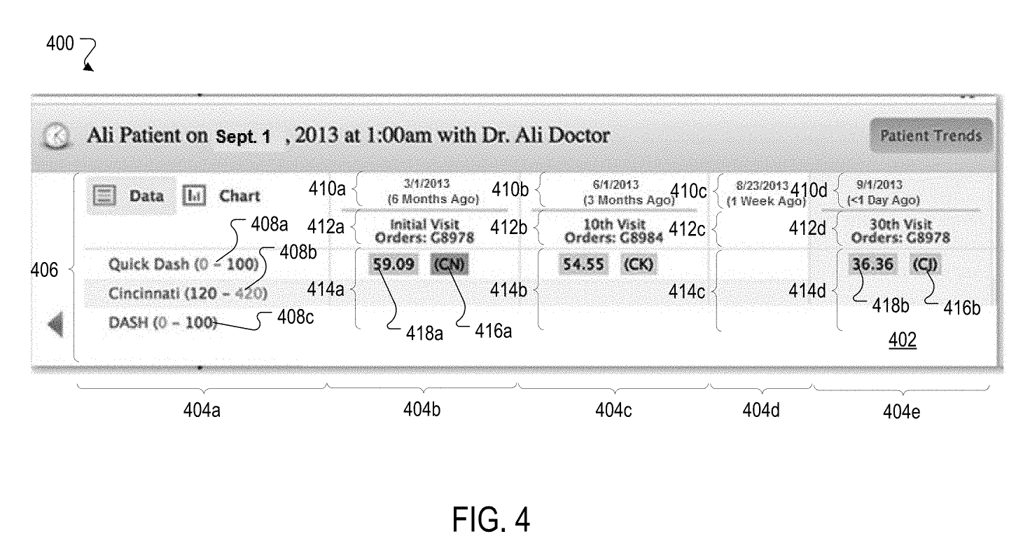

FIG. 4 is a screen image of a graphical user interface 400 that can be used to present information to a medical professional regarding a particular user (e.g., medical patient). In general, the graphical user interface 400 can present information indicative of progress that that user is making in his or her recovery. In some implementations, the graphical user interface 400 is presented as an appointment, although the information presented in the graphical user interface 400 can be presented using other workflows besides managing appointments. In general, the graphical user interface includes a table 402 that can be used to present information indicative of the progress that use is making.

In general, the table 402 can include a number of columns 404a-404e that can be used to present information indicative of the progress that a particular user in making. In some implementations, the table 402 may include multiple rows, similar to row 406 for one or more users that frequent the service provider.

Column 404a can present information indicative of one or more outcome questionnaires that are provided by the service provider. In an example, the graphical user interface 400 can indicate that a “Quick Dash” outcome questionnaire can be provided, that a “Cincinnati” outcome questionnaire can be provided, and that a “DASH” outcome questionnaire can be provided. In some implementations, color-coded ranges of outcome scores can also be presented. In a particular example, the indication 408a for the “Quick Dash” outcome questionnaire is presented with a color-coded range from zero to one-hundred, where zero is indicated as being an outcome score associated with the least impairment (e.g., based on the green-colored text) and one-hundred is indicated as being an outcome score associated with the most impairment (e.g., based on the red-colored text). As another particular example, the indication 408b for the “Cincinnati” outcome questionnaire is presented with a color-coded range from one-hundred and twenty to four-hundred and twenty, where one-hundred and twenty is indicated as being an outcome score associated with the most impairment (e.g., based on the red-colored text) and four-hundred and twenty is indicated as being associated with the least impairment (e.g., based on the green-colored text).

Each of columns 404b-404e can present information indicative of a particular visit to the service provider. In general, each of the columns 404b-404e include a date region 410a-410d, an orders region 412a-412d, and an outcome score and modifier region 414a-414d, respectively.

In general, the date region 404a-410d includes information indicative of when a user visited the service provider. In an example, column 404b includes a date region 410a that presents information indicative of a visit on Mar. 1, 2013 and that the visit was 6 months ago.

In general, the orders region 412a-412d includes information indicative of a code (e.g., such as a G-code) to be provide to an insurance provider. In some implementations, the status report and corresponding information included in the orders region 412a-412d is based on the type of visit (e.g., initial visit, tenth, visit, and so forth) and outcome score determined for a particular outcome questionnaire provided at the time of the particular visit. In an example, column 404b includes an orders region 412a that presents information indicative of an initial visit and a G-code of G8978 that is indicative of a mobility impairment at the onset of therapy. In general, the G-code can be determined by a rules engine, e.g., based a type of visit. Additional G-codes and the rules engine are described in more detail below.

In general, the outcome score and modifier region 414a-414d includes an outcome score and a modifier determined from the outcome score that are based on answers provided by the user when answering an outcome questionnaire. In an example, column 404b includes a modifier 416a “(CN)” that is associated with an outcome score 418a of “59.09.” In some implementations, the modifier and outcome score can be color-coded to indicate a level of severity and/or whether the modifier and outcome score meet or exceed a goal level of impairment that is set by the serve provider after a user's initial visit. In an example, the modifier 416a is color-coded red to indicate a higher level of impairment relative to the modifier 416b that is color-coded green (or green-ish). Likewise, in some implementations, the outcome score 418a is color-coded yellow to indicate a higher level of impairment relative to the outcome score 418b that is cored green (or green-ish).

In some implementations, the regions 410, 412, and 414, can be left blank to indicate that an outcome questionnaire was not provided to the user or that the user did not provide answers to the outcome questionnaire. In a particular example, column 404d includes regions 410c, 412c, and 414c that are blank, which may indicate that an outcome assessment questionnaire was not provided at that particular visit to the service provider.

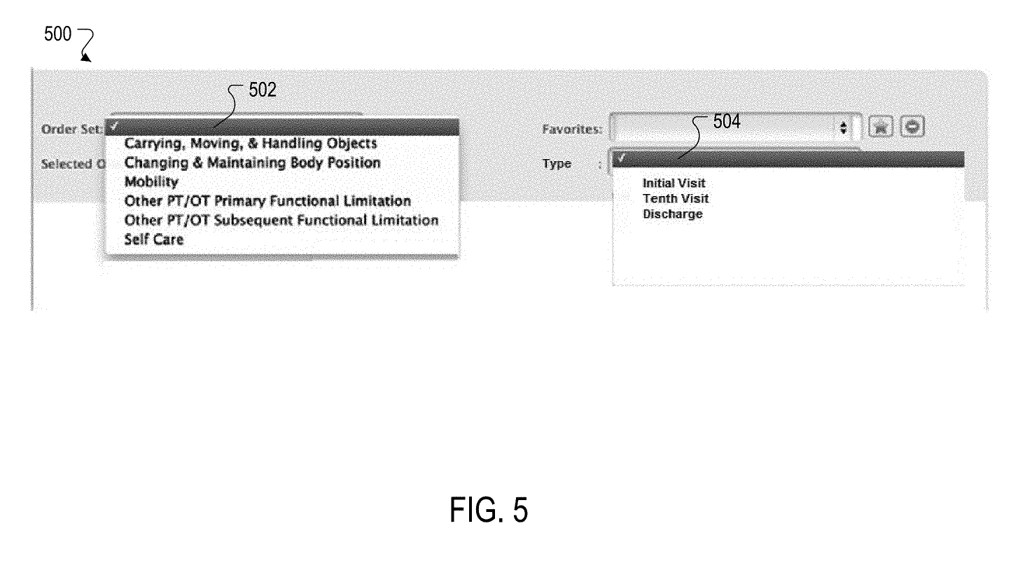

FIG. 5 is a screen image of a graphical user interface 500 that be used to generate a code, such as a G-code, that can be provided in an insurance report or other graphical user interface (e.g., graphical user interface 400 (FIG. 4)). In general, the graphical user interface 500 includes an order set user interface control 502 and a type of visit user interface control 504. In some implementations the controls 502 and 504 are drop-down controls. For simplicity, the graphical user interface 500 is illustrated showing both user interface controls 502 and 504 presenting one or more elements in their respective drop-down controls, although in general, each of the controls 502 and 504 present one or more of their respective elements independently in response to receiving user selection of the particular control.

The order set user interface control 504 can present information indicative of a type of functional limitation. In a particular example, the functional limitations that can be presented by the order set user interface control 504 include a mobility functional limitation, a changing and maintaining body position functional limitation, a carrying, moving and handling objects functional limitation, a self care functional limitation, a other physical or occupational primary functional limitation, a other physical or occupational subsequent functional limitation, a swallowing functional limitation, a motor speech functional limitation, a spoken language comprehension functional limitation, a spoken language expression functional limitation, an attention functional limitation, a memory functional limitation, a voice functional limitation, a other speech language pathology functional limitation, or other limitations.

The type of visit user interface control 504 can present information indicative of a type of visit. In an example, the type of visit that can be presented by the type of visit user interface control 504 can include an initial visit, a tenth visit, a discharge visit, and other types of visits.

Once a medical professional or other member of the service provider selects a functional limitation and a type of visit, a system (e.g., server 112 (FIG. 1)) can generate a code (e.g., a G-code) based on the selected functional limitation and visit type. In a particular example, the G-code can be provided in a graphical user interface (e.g., graphical user interface 400 (FIG. 4)) or an insurance report, accordingly to a selection made by the medical professional. For example, if the medical professional wishes to generate an insurance report the generated code can be included in the insurance report.

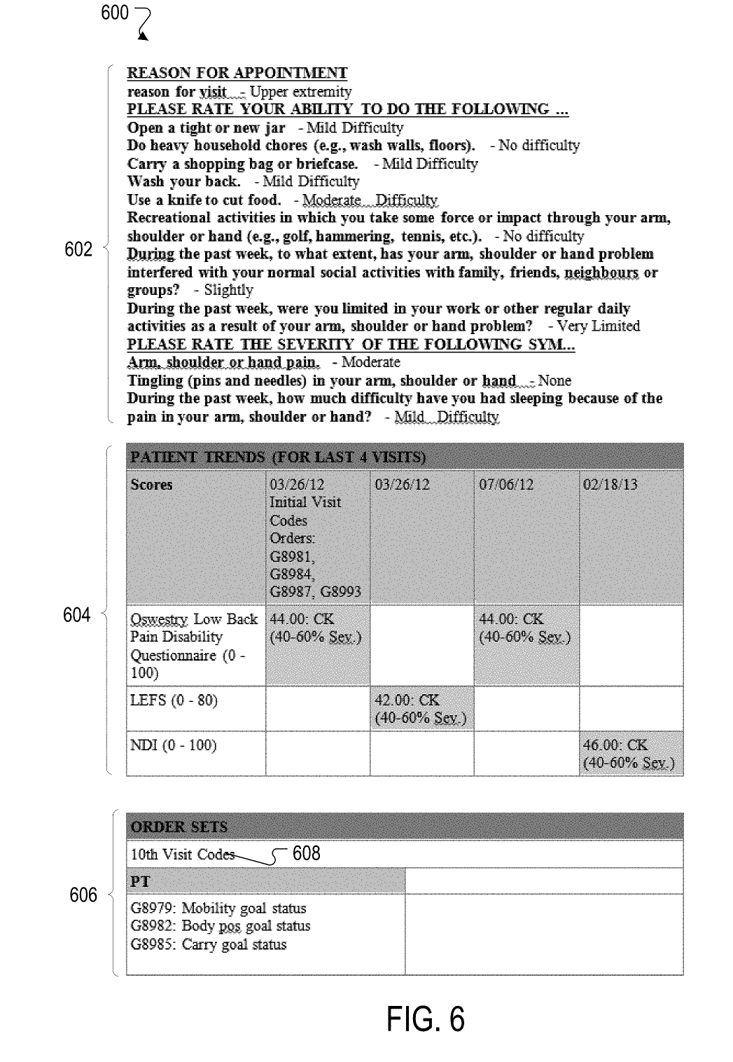

FIG. 6 is an illustration of an example insurance report 600. In general, the insurance report 600 can be generated in response to a system receiving a request from a medical professional or other member associated with the service provider. In an example, the insurance report 600 includes multiple regions that can be used by an insurance provider to track or otherwise validate the care that the particular user is receiving and to pay the service provider for the medical services rendered. In a particular example, the insurance report includes a question and answer region 602, a user trends region 604, and an order sets region 606.

The question and answer region 602 includes one or more questions of a particular outcome questionnaire and their associated answers as provided by the user (e.g., medical patient). In an example, the question and answer region 602 includes both the actual text of the question(s) and the actual text of the answers that were provided by the user. In some implementations, the text can be modified to indicate which text pertains to a question and which text pertains to an answer. In a particular example, the questions are presented using a bold font and the answers are presented using a normal or un-bolded font.

The user trends region 604 includes information indicative of past visits of the user (e.g., the medical patient) based on one or more outcome answers that were provided by the user in response to the outcome questionnaire. In some implementations, the user trends region 604 can provide similar information to that of the graphical user interface 400 (FIG. 4).

For example, the user trends region 604 can present information indicative of outcome questionnaires and associated outcome score ranges (e.g., similar to column 404a). As another example, the user trends region 604 can present information indicative of one or more outcomes. In a particular example, the user trends region 604 can present information indicative of when the user previously visited the service provider (e.g., similar to regions 410a-410d in columns 404b-404e). In another particular example, the user trends region 604 can present information indicative of a code (e.g., similar to regions 412a-412d in columns 404b-404e). In another particular example, the user trends region 604 can present information indicative of an outcome score and a modifier determined from the outcome score that are based on answers provided by the user when answering an outcome questionnaire (e.g., similar to regions 414a-414d in columns 404b-404e).

The order sets region 606 includes information indicative of a code and type of visit based on the particular visit that prompted the generation of the insurance report. In a particular example, as indicated by the order sets region 606, the user has attended his tenth visit (e.g., as shown by text region 608). Based on the answers provided by the user during that visit and because the visit is a tenth visit one or more G-codes associated with one or more types of functional limitation may be included in the insurance report 600. That is because, in some implementations, a tenth visit matches a reporting interval the G-codes that are determined pertain to the reporting interval and not, e.g., an initial visit or discharge visit.

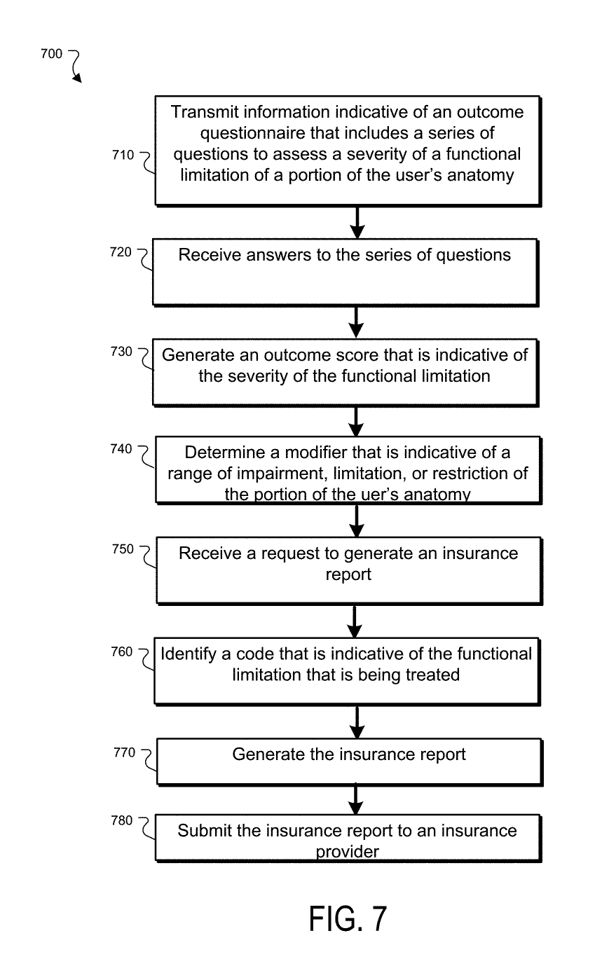

FIG. 7 is a flow chart of an example process 700 that can be used to generate and submit an insurance report. In general, the information contained within the insurance report can be based on information provided by a user that is indicative of a functional limitation of the user's anatomy. In general, the process 700 is described in relation to a server 112 (FIG. 1) that is configured to perform the process 700, although other systems may be configured to perform the process 700.

In operation, the server 112 transmits (710), to a first client device of a user, information indicative of an outcome questionnaire, with the outcome questionnaire comprising a series of questions to assess a severity of a functional limitation of a portion of the user's anatomy. In some implementations, the functional limitation can be one or more of a mobility functional limitation, a changing and maintaining body position functional limitation, a carrying, moving and handling objects functional limitation, a self care functional limitation, a other physical or occupational primary functional limitation, a other physical or occupational subsequent functional limitation, a swallowing functional limitation, a motor speech functional limitation, a spoken language comprehension functional limitation, a spoken language expression functional limitation, an attention functional limitation, a memory functional limitation, a voice functional limitation, and a other speech language pathology functional limitation.

The server 112 receives (720), from the first client device, answers to the series of questions. In an example, a user of the first client device can provide touch-input to select particular answers to the series of questions.

The server 112 generates (730), based on the answers, an outcome score that is indicative of the severity of the functional limitation. In some implementations, the server 112 can access a rules engine that comprises a plurality of rules for generating outcome scores, based on answers to questions included in outcome questionnaires. The server 112 can also cause an execution of the rules engine. The server 112 can apply, based on execution of the rules engine, one or more of the rules in the plurality to the received answer. The server 112 can also generate, based on applying, the outcome score.

The server 112 determines (740), at least partly based on the outcome score, a modifier that is indicative of a range of impairment, limitation, or restriction of the portion of the user's anatomy. In an example, the server 112 can communicate with a rules engine that can determine a modifier based on a range of impairment, limitation, or restriction of the portion of the user's anatomy. In some implementations, the rules engine can access a mapping when determining a modifier based on the range of impairment. A mapping that can be used is described in more detail elsewhere in this specification.

The server 112 receives (750), from a second client device of a service provider, a request to generate an insurance report, with the request comprising a selected type of functional limitation and a visit type. In an example, the second client device can present graphical user interface 500 (FIG. 5) to a medical professional or other member of the service provider. In some implementations, the visit type can be one or more of an initial visit, a tenth visit and a discharge visit.

The server 112 identifies (760), based on contents of the received request, a code that is indicative of the functional limitation that is being treated by the service provider and that is indicative of a point in time in a course of treatment of the user. In some implementations, the code is a G-code. Particular G-codes are described elsewhere in this specification.

The server 112 generates (770), based on the identified code and the modifier, the insurance report. In some implementations, the server 112 can also access a rules engine that comprises a plurality of rules for generating modifiers, with the rules engine including a plurality of rules specifying relationships among outcome scores and modifiers. The server 112 can also cause an execution of the rules engine. The server 112 can also apply, based on execution of the rules engine, one or more of the rules in the plurality to the generated outcome score. The server can also identify, based on applying, the modifier. In a particular example, the server 112 can identify a modifier by accessing table or other mapping of outcome scores and modifiers. In this example, server 112 identifies in the mapping an association between a particular outcome score or range of outcome scores and a modifier. An example mapping is shown in the following table:

TABLE 1

| Outcome Score | Modifier |
|---|---|
| 0 | CH |
| 1-29 | CI |
| 20-39 | CJ |
| 40-59 | CK |
| 60-79 | CL |
| 80-99 | CM |
| 100 | CN |

In some implementations, the insurance report generated by the server is a claim submission. In an example, the insurance report can be generated as part of a claim submission workflow when the service provider requests payment for medical services rendered for a particular user (e.g., medical patient).

The server 112 submits (780) the insurance report to an insurance provider. In an example, an insurance report (e.g., insurance report 600 (FIG. 6) can encoded as electronic document and sent by server 112 to another system operated by a particular insurance provider using network 110 (FIG. 1).

In some implementations, the server 112 can access information indicative of a mapping of items of code selection information to a plurality of codes. In a particular example, an item of code selection information may include information specifying a type of functional limitation and information specifying a visit type. The server 112 may also determine a match between (i) contents of the item of code selection information, and (ii) the selected type of functional limitation and the visit type specified in the request. In a particular example, identifying the code includes the server 112 identifying, based on the determined match, a code associated with the matching item of code selection information.

In an example, a code (e.g., a G-code) is specific to a type of functional limitation and a visit type. For example, as shown in the below Table 1, the “walking & moving around functional limitation” has three different G-codes, e.g., one G-code for visit types of “at therapy episode outset and at reporting intervals,” another G-code for visit types of “at therapy episode outset, at reporting intervals, and at discharge or to end reporting,” and still another G-code for “at discharge from therapy or to end reporting.” In this example, server 112 receives a request for an insurance report. The request includes the functional limitation that is being treated by the service provider and that is indicative of a point in time in a course of treatment of the user. Based on the contents of the request, server 112 identifies a code that is associated with the functional limitation that is being treated by the service provider and that is indicative of the point in time in a course of treatment of the user. For example, the request may include walking & moving around—at therapy episode outset and at reporting intervals. Based on the contents of the request, server 112 may perform a table lookup to look-up (and to select) (e.g., in Table 1—which may be stored in a data repository) a G-code (e.g., G8978) that is associated with code selection information of walking & moving around—at therapy episode outset and at reporting intervals.

In a particular example, the server 112 can identify a code associated with the matching item of code selection information based on the following table:

TABLE 2

| G-Code | Code Selection Information |
|---|---|
| G8978 | Mobility: walking & moving around functional limitation, current status, at |
| | therapy episode outset and at reporting intervals |
| G8979 | Mobility: walking & moving around functional limitation, projected goal |
| | status, at therapy episode outset, at reporting intervals, and at discharge or to |
| | end reporting |
| G8980 | Mobility: walking & moving around functional limitation, discharge status, at |
| | discharge from therapy or to end reporting |
| G8981 | Changing and maintaining body position functional limitation, current status, |
| | at therapy episode outset and at reporting intervals |
| G8982 | Changing and maintaining body position functional limitation, projected goal |
| | status, at therapy episode outset, at reporting intervals, and at discharge or to |
| | end reporting |
| G8983 | Changing and maintaining body position functional limitation, discharge |
| | status, at discharge from therapy or to end reporting |
| G8984 | Carrying, moving and handling objects functional limitation, current status, at |
| | therapy episode outset and at reporting intervals |
| G8985 | Carrying, moving and handling objects functional limitation, projected goal |
| | status, at therapy episode outset, at reporting intervals, and at discharge or to |
| | end reporting |
| G8986 | Carrying, moving & handling objects functional limitation, discharge status, at |
| | discharge from therapy or to end reporting |
| G8987 | Self care functional limitation, current status, at therapy episode outset and at |
| | reporting intervals |
| G8988 | Self care functional limitation, projected goal status, at therapy episode outset, |
| | at reporting intervals, and at discharge or to end reporting |
| G8989 | Self care functional limitation, discharge status, at discharge from therapy or to |
| | end reporting |
| G8990 | Other physical or occupational primary functional limitation, current status, at |
| | therapy episode outset and at reporting intervals |
| G8991 | Other physical or occupational primary functional limitation, projected goal |
| | status, at therapy episode outset, at reporting intervals, and at discharge or to |
| | end reporting |
| G8992 | Other physical or occupational primary functional limitation, discharge status, |
| | at discharge from therapy or to end reporting |
| G8993 | Other physical or occupational subsequent functional limitation, current status, |
| | at therapy episode outset and at reporting intervals |
| G8994 | Other physical or occupational subsequent functional limitation, projected goal |
| | status, at therapy episode outset, at reporting intervals, and at discharge or to |
| | end reporting |
| G8995 | Other physical or occupational subsequent functional limitation, discharge |
| | status, at discharge from therapy or to end reporting |
| G8996 | Swallowing functional limitation, current status at time of initial therapy |
| | treatment/episode outset and reporting intervals |
| G8997 | Swallowing functional limitation, projected goal status, at initial therapy |
| | treatment/outset and at discharge from therapy |
| G8998 | Swallowing functional limitation, discharge status, at discharge from |
| | therapy/end of reporting on limitation |
| G8999 | Motor speech functional limitation, current status at time of initial therapy |
| | treatment/episode outset and reporting intervals |
| G9186 | Motor speech functional limitation, projected goal status at initial therapy |
| | treatment/outset and at discharge from therapy |
| G9158 | Motor speech functional limitation, discharge status at discharge from |
| | therapy/end of reporting on limitation |
| G9159 | Spoken language comprehension functional limitation, current status at time of |
| | initial therapy treatment/episode outset and reporting intervals |
| G9160 | Spoken language comprehension functional limitation, projected goal status at |
| | initial therapy treatment/outset and at discharge from therapy |
| G9161 | Spoken language comprehension functional limitation, discharge status at |
| | discharge from therapy/end of reporting on limitation |
| G9162 | Spoken language expression functional limitation, current status at time of |
| | initial therapy treatment/episode outset and reporting intervals |
| G9163 | Spoken language expression functional limitation, projected goal status at |
| | initial therapy treatment/outset and at discharge from therapy |
| G9164 | Spoken language expression functional limitation, discharge status at discharge |
| | from therapy/end of reporting on limitation |
| G9165 | Attention functional limitation, current status at time of initial therapy |
| | treatment/episode outset and reporting intervals |
| G9166 | Attention functional limitation, projected goal status at initial therapy |
| | treatment/outset and at discharge from therapy |
| G9167 | Attention functional limitation, discharge status at discharge from therapy/end |
| | of reporting on limitation |
| G9168 | Memory functional limitation, current status at time of initial therapy |
| | treatment/episode outset and reporting intervals |
| G9169 | Memory functional limitation, projected goal status at initial therapy |
| | treatment/outset and at discharge from therapy |
| G9170 | Memory functional limitation, discharge status at discharge from therapy/end |
| | of reporting on limitation |
| G9171 | Voice functional limitation, current status at time of initial therapy |
| | treatment/episode outset and reporting intervals |
| G9172 | Voice functional limitation, projected goal status at initial therapy |
| | treatment/outset and at discharge from therapy |
| G9173 | Voice functional limitation, discharge status at discharge from therapy/end of |
| | reporting on limitation |
| G9174 | Other speech language pathology functional limitation, current status at time of |
| | initial therapy treatment/episode outset and reporting intervals |
| G9175 | Other speech language pathology functional limitation, projected goal status at |
| | initial therapy treatment/outset and at discharge from therapy |
| G9176 | Other speech language pathology functional limitation, discharge status at |
| | discharge from therapy/end of reporting on limitation |

In some implementations, the outcome score includes a first outcome score. In an example, the server 112 can generate an insurance report that includes a first visual representation of a timeline that specifies at least two visits of the user to the service provider. In a particular example, the insurance report 600 (FIG. 6) generated by the server 112 includes visit on Mar. 26, 2012, Jul. 6, 2012, and Feb. 18, 2013. The insurance report may also include a second visual representation of the first outcome score for the outcome questionnaire at a first one of the at least two visits. In a particular example, the insurance report 600 generated by the server includes an outcome score of 44.00 for an initial visit on Mar. 26, 2012 for the Oswestry Low Back Pain Disability Questionnaire. The insurance report may also include a third visual representation of a second outcome score for the outcome questionnaire at a second one of the at least two visits. In an example, the insurance report 600 generated by the server 112 includes an outcome score of 44.00 for a subsequent visit on Jul. 6, 2012 for the Oswesrty Low Back Pain Disability Questionnaire.

In some implementations, the server 112 can receive, from the first client device, information indicative of the portion of the user's anatomy in which the user is experiencing the functional limitation. In an example, the server 112 can receive a selection of a particular part of the user's anatomy from one of client devices 102 and 108 in response to presenting a question such as “State the reason for your visit,” or a similar question designed to elicit an anatomy-based response from the user. In an example, the server 112 can select, from a plurality of outcome questionnaires and based on the received information, the outcome questionnaire that is submitted to the first client device, with the selected outcome questionnaire including questions that pertain to the portion of the user's anatomy in which the user is experiencing the functional limitation. In an example, the server 112 may access a data repository (e.g., data repository 114 (FIG. 1)) that specifies a mapping between a particular portion of anatomy and one or more appropriate outcome questionnaires that can be provided to the user.

Embodiments can be implemented in digital electronic circuitry, or in computer hardware, firmware, software, or in combinations thereof. An apparatus can be implemented in a computer program product tangibly embodied or stored in a machine-readable storage device for execution by a programmable processor; and method actions can be performed by a programmable processor executing a program of instructions to perform functions by operating on input data and generating output. The embodiments described herein, and other embodiments of the invention, can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object oriented programming language, or in assembly or machine language if desired; and in any case, the language can be a compiled or interpreted language.

Processors suitable for the execution of a computer program include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Computer readable media for embodying computer program instructions and data include all forms of non-volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in special purpose logic circuitry. Any of the foregoing can be supplemented by, or incorporated in, ASICs (application-specific integrated circuits).

To provide for interaction with a user, embodiments can be implemented on a computer having a display device, e.g., a LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments can be implemented in a computing system that includes a back end component, e.g., as a data server, or that includes a middleware component, e.g., an application server, or that includes a front end component, e.g., a client computer having a graphical user interface or a Web browser through which a user can interact with an implementation of embodiments, or any combination of such back end, middleware, or front end components. The components of the system can be interconnected by any form or medium of digital data communication, e.g., a communication network. Examples of communication networks include a local area network (LAN) and a wide area network (WAN), e.g., the Internet.

The system and method or parts thereof may use the “World Wide Web” (Web or WWW), which is that collection of servers on the Internet that utilize the Hypertext Transfer Protocol (HTTP). HTTP is a known application protocol that provides users access to resources, which may be information in different formats such as text, graphics, images, sound, video, Hypertext Markup Language (HTML), as well as programs. Upon specification of a link by the user, the client computer makes a TCP/IP request to a Web server and receives information, which may be another Web page that is formatted according to HTML. Users can also access other pages on the same or other servers by following instructions on the screen, entering certain data, or clicking on selected icons. It should also be noted that any type of selection device known to those skilled in the art, such as check boxes, drop-down boxes, and the like, may be used for embodiments using web pages to allow a user to select options for a given component. Servers run on a variety of platforms, including UNIX machines, although other platforms, such as Windows 2000/2003, Windows NT, Sun, Linux, and Macintosh may also be used. Computer users can view information available on servers or networks on the Web through the use of browsing software, such as Firefox, Netscape Navigator, Microsoft Internet Explorer, or Mosaic browsers. The computing system can include clients and servers. A client and server are generally remote from each other and typically interact through a communication network. The relationship of client and server arises by virtue of computer programs running on the respective computers and having a client-server relationship to each other.

Other embodiments are within the scope and spirit of the description claims. For example, due to the nature of software, functions described above can be implemented using software, hardware, firmware, hardwiring, or combinations of any of these. Features implementing functions may also be physically located at various positions, including being distributed such that portions of functions are implemented at different physical locations. The use of the term “a” herein and throughout the application is not used in a limiting manner and therefore is not meant to exclude a multiple meaning or a “one or more” meaning for the term “a.” Additionally, to the extent priority is claimed to a provisional patent application, it should be understood that the provisional patent application is not limiting but includes examples of how the techniques described herein may be implemented.

A number of exemplary embodiments of the invention have been described. Nevertheless, it will be understood by one of ordinary skill in the art that various modifications may be made without departing from the spirit and scope of the invention.

What is claimed is:

1. A system for searching data structures stored in a database and for processing a plurality of structured data items with a rules engine that applies programmed logic to one or more data structures identified from the searching and to one or more of the structured data items in the plurality, the data processing system comprising:
a database for storing a plurality of data structures;
a rules engine that comprises programmed logic that defines rules for (i) generating an outcome score and that receives structured data items representing a plurality of answers responsive to a set of questions, wherein each answer of the plurality of answers is received from a client device in response to a particular question in the set of questions, wherein the rules engine performs operations comprising
executing one or more rules of the rules engine against the received structured data items to generate an outcome score;
accessing, from the database and by the rules engine, a data structure that maps outcome scores to attribute modifiers;
searching the accessed data structure to identify a first entry that is mapped to a second entry representing the generated outcome score;
determining, by the rules engine and based on contents of the identified first entry, an attribute modifier; and
accessing, in memory, another data structure that specifies one or more values related to the attribute modifier that are indicative of treatment continuation.
